(19) United States
(12) Patent Application Publication
(10) Pub. No.: US 2007/0148241 A1
(43) Pub. Date: Jun. 28, 2007

(54) Use of an intermediate for controlling the release profile of an oral formulation of a medicament

(75) Inventors: Michael Braun, Senden (DE); Klaus Neuer, Schwendi-Orsenhausen (DE); Thomas Beckert, Warthausen-Birkenhard (DE); Carmen Heinz, Schwendi-Horenhausen (DE); Melanie Brunner, Obermarchtal (DE)

Correspondence Address: RATNERPRESTIA, P O BOX 980, VALLEY FORGE, PA, 19482-0980, US

(73) Assignee: Oramon Arzneimittel GmbH & Co. KG, Laupheim (DE)

(21) Appl. No.: 11/506,382

(22) Filed: Aug. 18, 2006

(30) Foreign Application Priority Data
Aug. 18, 2005 (EP) 05 017 958.9

(51) Int. Cl. A61K 9/24

(52) U.S. Cl. 424/472

(57) ABSTRACT

A swellable intermediate layer that includes a swellable material selected from the group of starch, starch derivatives such as pregelatinised starch, sodium carboxyethyl starch, sodium starch gycolate, cellulose, cellulose ethers such as HPMC, HPC, HEC MC, sodium carboxymethyl cellulose, carrageenans, aginates, pectins, xanthanes and their derivatives, guar gum, tragacanth, polyvinyl pyrrolidones and mixtures thereof, in a delayed-release, solid pharmaceutical formulation for peroral use. The formulation has a substrate containing an active substance, the swellable intermediate layer and a retarding coating layer for the adjustment of a release profile for the active substance which essentially permits complete release near the end.

The present invention relates to the use of a swellable intermediate layer made of a swellable material for the adjustment of a release profile of a delayed release, solid pharmaceutical formulation for peroral use, as well as delayed release, solid pharmaceutical formulations for peroral use containing such a swellable intermediate layer.

### PRIOR ART

Delayed release, solid pharmaceutical formulations for oral use are well known. They are widely used in pain therapy, for cardiovascular diseases or diseases of the central nervous system, among other things. The purpose of the administration of these delayed (i.e. slow-release) formulations is to realise a sustained pharmacological effect after administration that is longer than is possible through the -administration of dosage forms that release instantly or immediately. Such longer time spans for the pharmacological effect offer numerous therapeutic advantages that cannot be realised with the conventional preparations that release instantly. The therapy can therefore be continued, for example, even without interrupting the patient's sleep. In addition, a reduction of the doses to be taken daily increases patient compliance considerably.

With the administration of immediate-release, fast-acting pharmaceutical forms, the administration must be carried out at regular, brief intervals of 4 to 6 hours, for example, in order to maintain a specific active substance level in the plasma of the patient.

Peaks of this blood level, as well as too great, a slump are to be avoided, which can be difficult in view of differential secretion, metabolic inactivation, or even patient compliance. Specifically, intake at very regular intervals is to therefore be ensured, for example. However, this is often not provided for.

The delayed release assists in overcoming these problems. In this context, active substance is released continuously over a long period from a dose taken once, which leads to appropriate plasma levels. Osmotic, eroding and diffusion systems, among others, can be distinguished according to the functional principle. In turn, the diffusion systems can be controlled by matrix or membrane. Hybrid forms are also possible. The delayed-release membrane-controlled diffusion systems are typically implemented through the application of a coating made of an insoluble polymer, made of ethyl cellulose for example, to which additional components such as plasticisers, for example, can be added. Corresponding solid, delayed-release oral dosage forms are described in EP-A-1 243 269 and EP-A-1 419 766, for example.

Both documents describe delayed-release opioid formulations in which the delayed release is typically achieved by means of extra layers of a substrate containing the active substance or (pellet) cores with a sufficient amount of an aqueous dispersion of an insoluble polymer such as ethyl cellulose, acryl polymer, shellac, zein, a hydrophobic wax and mixtures of them for obtaining a cover coat. Adjuvants such as plasticisers (for example, citrate ester, phthalate ester or sebacate ester), dyestuffs, pore-forming agents (such as hydroxypropyl methyl cellulose) or other hydrophilic polymers, the agents that support erosion (such as starch), or similar agents can be added to this coating.

In addition, the pharmaceutical formulations according to EP 1 243 269 and EP 1 419 766 can contain a barrier layer between the substrate that contains the active substance and the coating layer that causes the delayed release. In the typical case, this serves to improve the stability of the active substance by separating the active substance from the coating layer that controls the delayed release. Such an intermediate layer preferably contains hydroxypropyl methylcellulose of low viscosity (Pharmacoat 603, for example). However, this barrier layer should not affect the release speed of the product.

The solid, delayed-release pharmaceutical formulations for oral administration known from the prior art have the disadvantage that their release characteristics are substantially dependent on the thickness of the delayed-release coating made. of hydrophobic polymer, in addition to the polymer characteristics and the type and the proportion of the adjuvants used. If it is chosen to be very thick, a clearly more limited active substance release/unit of time compared to the beginning, which means a flattening of the release profile, is often observed by the end of the test period, which is normally between 8 and 12 hours. As a result, in many cases no complete active substance release can be achieved. In addition, the initial release rate can turn out to be too low. If the thickness of the coating is reduced correspondingly, the release is often too rapid at the beginning of the test period (up to 1 hour after administration, for example). In other words, the release profile that can be achieved using such delayed-release coatings has a too flat gradient by the end of the test period.

To solve this problem, the documents specified above recommend the use of certain agents that modify the release in the coating layer such as pore-forming agents in the form of hydrophilic soluble polymers, long-chain hydrocarbons that can be metabolised such as fatty acids, fatty alcohols, glycerin esters themselves, polyalkylene glycol, starches, gums, polyesters or similar agents. However, the release profile achieved by doing so always still shows a release of approximately 12.5% to approximately 42.5% after one hour and more than approximately 60% after 8 hours, measured using the USP paddle method at 100 rpm and 900 ml of aqueous buffer (pH between 1.6 and 7.2) at 37° C. As before, in doing so essentially no complete release is achieved by the end of the release period, but an effective delay of the release is considerably impaired even at the start in part. The initial insufficient release for thick coatings can be made up for through the use of mixtures of delayed-release and immediate-release pharmaceutical forms. However, 2 profiles are superimposed, and the relationship of both forms must be coordinated carefully.

The object of the present invention is to overcome these and other disadvantages of the prior art.

### BRIEF DESCRIPTION OF THE INVENTION

The present invention should overcome the above problems from the prior art, and in particular allow for control or adjustment of the release profile of a solid, delayed-release pharmaceutical formulation for peroral use. This is made possible through the use of a swellable intermediate layer that includes a swellable material selected from the group that comprises starch, starch derivatives such as pregelatinised starch, sodium carboxyethyl starch, sodium starch glycolate, cellulose, cellulose ethers such as HPMC, HPC, HEC, MC, sodium carboxymethyl cellulose, carrageenans, alginates, pectins, xanthanes and their derivatives, guar gum, tragacanth, polyvinyl pyrrolidones and mixtures thereof, in a delayed-release, solid pharmaceutical formulation for peroral use, with said formulation having a substrate containing an active substance, the swellable intermediate layer and a retarding coating layer for the adjustment of a release profile for the active substance which essentially permits complete release by the end.

In exactly the same way, the present invention relates to a delayed-release, solid pharmaceutical formulation for peroral use, with said formulation having a substrate that contains an active substance, a swellable intermediate layer and a retarding coating layer, wherein the swellable intermediate layer includes a swellable material that is selected from the group that comprises starch, starch derivatives such as pregelatinised starch, sodium carboxyethyl starch, sodium starch glycolate, cellulose, HPC, HEC, MC, sodium carboxymethyl cellulose, carrageenans, alginates, pectins, xanthanes and their derivatives, guar gum, tragacanth, polyvinyl pyrrolidones and mixtures thereof, and which is available in an effective amount for the adjustment of a release profile for the active substance which essentially permits complete release by the end.

### MORE SPECIFIC DESCRIPTION OF THE INVENTION

The present invention is based on the knowledge that the release profile of delayed-release solid pharmaceutical formulations for peroral use cannot be controlled exclusively through the selection and design of the retarding coating layer, but can also be controlled through the use of a special intermediate layer, namely a swellable intermediate layer.

In the sense of the present application, “delayed-release” means a pharmaceutical formulation in which the active substance contained in it is essentially completely released within a time span of more than 6 hours, preferably more than 8 hours, and typically 8 to 12 hours after peroral administration. According to the invention, the release is measured based on the USP paddle method or the rotating basket method, as is described in the US Pharmacopoeia XXII (1990). A paddle apparatus with rotating baskets according to the European Pharmacopoeia 2.9.3-1 is used in this case. It is rotated over the measurement period at 100 rpm in 900 ml of a phosphate buffer, pH 6.4 according to the USP, and the amount of active substance released into the solution is determined, whereby the release is specified as a percent of the total amount contained in the formulation:
Released amount×100/total amount=% release

According to the invention, “essentially complete release” means a release of at least 75% of the total amount of the active substance contained in the formulation, preferably at least 85% of the total amount of the active substance contained in the formulation. “By the end” of the release means the endpoint of the above time span of more than 6 hours, and preferably 8 to 12 hours after administration. The “initial” release relates to the release within 1 hour after administration.

The use of the swellable intermediate layer according to the invention in the delayed-release, solid pharmaceutical formulation for peroral use, which has a substrate that contains an active substance, the swellable intermediate layer and a retarding coating layer, permits the adjustment of the release profile of the active substance from the pharmaceutical formulation such that an essentially complete release is possible by the end of the release (and therefore after approximately 8 hours) without substantially affecting the initial release. In other words, a steeper gradient for the release profile in comparison to a corresponding formulation without the intermediate layer is achieved through the use of the swellable intermediate layer. This means that, by the end, the amount of active substance released from the pharmaceutical formulation is substantially greater than the amount that is released from a corresponding formulation without the intermediate layer.

In addition, the swellable intermediate layer can modulate the release profile such that the release is initially retarded or reduced in comparison to that for the corresponding formulation without the intermediate layer. An even steeper release profile can be achieved by doing so.

After 8 hours and measured based on the rotating basket method according to the European Pharmacopoeia 2.9.3-1, the release preferably amounts to at least 75% of the total amount of the active substance contained in the formulation, more strongly preferred at least 85% of the total amount, and most preferred at least 90% of the total amount of the active substance contained in the formulation. In addition, after 8 hours the released amount of active substance preferably amounts to at least 130% of the amount that is released from the corresponding formulation without the intermediate layer in the same period, again measured. based on the rotating basket method according to the European Pharmacopoeia 2.9.3-1. More strongly preferred, this amount is at least 150%, and even more strongly preferred at least 180% of the amount released from the corresponding formulation without the intermediate layer. All percentage information provided here is weight/weight.

Also preferred is a release profile in which the initial release after 1 hour, likewise measured based on the rotating basket method as above, amounts to between 5% and 35% (weight/weight) of the total amount of the active substance contained in the formulation. More strongly preferred, this amount is 15% to 25% of the total amount of active substance. In terms of the amount that is released by the corresponding formulation without the intermediate layer, the amount of active substance initially released after 1 hour, likewise measured based on the rotating basket method, preferably amounts to 70% to 120%, even more preferably 90% to 120%.

In a preferred embodiment, the pharmaceutical formulation has a release profile measured based on the rotating basket method, as a percentage of the total amount of the active substance contained in the formulation as follows:

| Time (h) | Percent |
|---|---|
| 1 | 5-35 |
| 3 | 45-75 |
| 8 | 85-100 |

In another embodiment, the pharmaceutical formulation has a release profile measured based on the rotating basket method, as a percentage of the total amount of the active substance contained in the formulation as follows:

| Time (h) | Percent |
|---|---|
| 1 | 5-15 |
| 3 | 25-35 |
| 8 | 70-80 |
| 12 | 85-95 |

The release profile above and advantageous embodiments of the invention can be achieved through the use of a swellable intermediate layer that comprises a swellable material. It is selected from the group that comprises starch, starch derivatives, cellulose, cellulose ethers, carrageenans, alginates, pectins, xanthanes and their derivatives, guar gum, tragacanth, polyvinyl pyrrolidones and mixtures thereof. To be mentioned in terms of starch derivatives in particular are pregelatinised starch, sodium carboxymethyl starch, sodium starch glycolate and similar agents. To be mentioned in terms of cellulose in particular ethers are hydroxypropyl methyl cellulose (HPMC), hydroxypropyl cellulose (HPC), hydroxyethyl cellulose (HEC), methyl cellulose (MC) and sodium carboxymethyl cellulose. Very particularly preferred are sodium carboxymethyl cellulose, sodium starch glycolate, cellulose ethers, here in particular hydroxypropyl methyl cellulose, sodium carboxymethyl cellulose and polyvinyl pyrrolidones, in particular cross-linked polyvinyl pyrrolidones.

The natural or synthetic polymers mentioned above are “swellable” in the sense of the invention because they can store water or another fluid such as salt solutions, alcohol solutions, gastric juice, etc. while increasing in volume. In doing so, the increase in volume amounts to at least 1.5 times the dry volume, and more strongly preferably at least 5-20 times. The polymers typically have a molecular weight in the range from 10,000 to 2,000,000. They can be cross-linked. Appropriate polymers can be obtained commercially. Suitable are, for example, the hydroxypropyl methyl cellulose product distributed under the name Pharmacoat 606 or 615, as well as the products that are distributed under the names Klucel EF, Klucel LF, Methocel A, Methocel E, Methocel F, Methocel K, Tylose H, Primojel, Ultramyl, Explotab, Ac-Di-Sol, Kollidon XL, Starch 1500 or Rhodigel.

The swellable intermediate layer can contain one or more adjuvants in addition to the swellable material, which are preferably selected from binding agents such as povidone, solvents such as water or alcohols and their mixtures, pH modulators, buffer substances, plasticisers, release agents and fillers and similar agents. The type and amount of the adjuvant or adjuvants is freely selectable so long as the swellable characteristics of the layer are not materially affected by them.

The swellable intermediate layer is applied to the substrate of the pharmaceutical formulation in an amount that is sufficient for the achievement of the desired effect on the release profile. At the same time, the amount of swellable intermediate layer that is applied to the substrate of the pharmaceutical formulation is dependent on the type of the swellable material as well as the type and thickness of the retarding coating layer. An optimisation for suitable amounts is within the ability of the person skilled in the art. Typically, the swellable intermediate layer is applied to the substrate of the pharmaceutical formulation in an amount of at least 0.01 mg/cm², more strongly preferably in an amount of 0.01 to 10 mg/cm², and most preferably in a range from 0.3 to 1.7 mg/cm².

The swellable intermediate layer can exist in the form of one or more swellable intermediate layers, whereby there can ultimately be a difference, for example, with respect to the swellable material or just the polymer length or the type and the availability of additives.

The retarding coating layer typically includes a retarding polymer. Each retarding polymer that delays the release and is known in the prior art can be used for this purpose. Suitable examples are ethyl cellulose, insoluble poly(meth)acrylates and/or polyvinyl acetates. Appropriate polymers and their solutions can also be obtained commercially in pharmaceutical quality. To be mentioned as examples are the polyacrylates such as Eudragit®NE, Eudragit®RS and Eudragit®RL as well as the polyvinyl acetate Kollicoat SR30D.

In addition to the retarding polymer, the retarding coating layer can include one or more adjuvants selected from the group that comprises binding agents, solvents such as water, alcohols and their mixtures, pH regulators, buffer substances, plasticisers, pore-forming agents, breakdown agents, retarding agents, additional film-forming agents, flavouring agents, dyestuffs, pigments, lubricants, additional modifiers for release such as gums, starches, oligosaccharides and similar agents, as well as release agents and fillers and similar agents. In addition, reference is made to EP-A-1 243 269 and EP-A-1 419 766 with respect to the usable retarding polymers and adjuvants. The type and amount of the adjuvants is freely selectable so long as the retarding characteristics of the coating layer are not materially affected.

The retarding film is typically applied at a film thickness of from 0.1 to 20 mg/cm², preferably 0.5 to 10 mg/cm² and even more strongly preferably 1 to 5.5 mg/cm². For the person skilled in the art, the selection of the thickness of the retarding coating layer obviously depends on the type of the retarding polymer selected, the desired delay of the release, and the type and amount of the adjuvants used. The adjustment of the corresponding characteristics of this coating layer is known from the prior art in the documents mentioned above. Its optimisation is within the ability of the person skilled in the art. The coating layer can be divided into multiple layers.

The solid, delayed-release pharmaceutical formulation for peroral administration according to the invention contains a substrate or core that contains the active substance(s), the intermediate layer(s) as described above, and a retarding coating layer. Additional layers, such as flavouring or optical improvement coating layers, and internal barrier layers can be present. The formulation is typically a formulation selected from the group that comprises tablets, capsules, pellets, microtablets, nanopellets, nanotablets, granulate, crystals as well as in capsules or sachets filled with pellets, microtablets, nanopellets, nanotablets, granulate and crystals. The pharmaceutical formulation preferably involves tablets, pellets, microtablets or capsules, and more strongly preferably pellets or microtablets. Pellets or microtablets preferably have a diameter (without coating) of 0.2 to 2.0 mm (preferably 0.7 to 1.2 mm), but can have a diameter of up to 5 mm. Small forms such as (nano)pellets, microtablets or nanotablets, crystals, powder or granulate can be pressed into disintegrating tablets.

The pharmaceutical formulation according to the invention has a substrate that contains the active substance. This substrate can contain the active substance as such or in combination with typical adjuvants. For example, the active substance can be processed into pellets with adjuvants through the use of extrusion, spheronisation or drip methods. The active substance can likewise be pressed into tablets or microtablets, or sprayed on appropriate tablet cores and pellet cores such as nonpareil pellets. Alternatively, the substrate can involve capsules already filled with active substance, for example in the form of pellets, powder, microtablets, nanopellets, nanotablets, granulate, crystals or similar agents, which in turn are provided according to the present invention with the intermediate layer and delayed-release layer.

In principle, the effect according to the invention for the control of the release profile for delayed-release, solid peroral pharmaceutical forms is applicable to all active substances that can be administered perorally. Preferably the active substance is selected from the group that comprises antihistamines (for example, dimenhydrinate, diphenhydramine, chlorpheniramine, cetirizine, loratadine, and dexchlorpheniramine maleate), analgesics (for example, hydromorphone, acetysalicylic acid, codeine, morphine, dihydromorphone, oxycodone, etc.), non-steroidal anti-inflammatories (for example, naproxen, diclofenac, indomethacine, ibuprofen, sulindac), antiemetics (for example, metoclopramide), antiepileptics (for example, phenytoine, meprobamate and nitrezepam), vasodilators (for example, nifedipine, papaverine, diltiazem and nicardirine), antitussive agents md expectorants (for example, codeine phosphate), antiasthmatics (for example, theophylline), spasmolytics (for example, atropine, scopolamine), agents for the treatment of diabetes (for example, insulin and oral anti-diabetics), diuretics (for example, ethacrynic acid, bendrofluazide), agents for the treatment of low blood pressure (for example, propranolol, clonidine), agents for the treatment of high blood pressure (e.g. clonidine, methyldopa), bronchodilatators (for example, albuterol), steroids (for example, testosterone, androgen, estradiol and its derivatives, hydrocortisone, triamcinolone, prednisone), antibiotics (for example, tetracycline), anti-hemorrhoidal agents, hypnotics, psychotropic agents, antidepressants (for example, amitriptyline, venlafaxine), agents for the treatment of diarrhoea, mucolytics, sedatives, decongestants, laxatives, vitamins and stimulants (including appetite suppressants such as phenylpropanolamine). More strongly preferably it involves an analgesic, an antidepressant or non-steroidal anti-inflammatory, and most preferable hydromorphone or amitriptyline.

The present invention also relates to delayed-release, solid pharmaceutical formulations for peroral use that are distinguished through the use of the swellable intermediate layer. The pharmaceutical formulations are as described above and in the claims.

Most preferably, pharmaceutical formulations are involved in the form of pellets with a diameter of 0.2 to 2 mm, which contain as an active substance hydromorphone or amitriptyline or pharmaceutically compatible salts thereof. According to the invention, the intermediate layer preferably comprises hydroxypropyl methyl cellulose or sodium carboxymethyl cellulose, with adjuvants as the case may be, whereby retarding films made of polyvinyl acetate and/or ethyl cellulose are used.

The following examples are to illustrate the invention, but not to limit it.

### EXAMPLE 1

Manufacture of the pellet core by means of an extrusion method. Intermediate layer and retarding film are placed on the fluidised bed. The pellets used have a diameter of 710-1200 μm.

Composition [mg/DF]

| | | | | Film | |
|---|---|---|---|---|---|
| | | [mg/DF] | [mg/DF] | thickness | |
| | Ingredients | A | B | mg/cm² | Function |
| Pellet core | Hydromorphone- | 16.0 | 16.0 | | Active substance |
| | HCl | | | | |
| | Avicel PH 101 | 163.0 | 163.0 | | Extrusion adjuvant |
| | Methocel E5 | 1.0 | 1.0 | | Binding agent |
| Intermediate layer | Pharmacoat 606 | 5.4 | — | 0.8 | Film-forming agent |
| Retarding film | Kollicoat SR30D | 25.2 | 25.2 | | Film-forming agent |
| | Triethyl citrate | 2.52 | 2.52 | 4.0 | Plasticiser |
| | Syloid 244 FP | 3.03 | 3.03 | | Release agent |
| | Kollidon 30 | 5.05 | 5.05 | | Film-forming agent |

In-vitro Release Profile

Paddle apparatus according to the European Pharmacopoeia 2.9.3-1, 100 rpm, phosphate buffer pH 6.4 according to the USP, 900 ml, n=6

| Time (h) | Average value [%] A | Average value [%] B |
|---|---|---|
| 1 | 31.6 | 25.2 |
| 2 | 57.5 | 43.4 |
| 3 | 71.8 | 53.2 |
| 4 | 82.0 | 58.5 |
| 5 | 86.5 | 62.5 |
| 6 | 90.2 | 65.4 |
| 7 | 93.4 | 68.2 |
| 8 | 95.3 | 70.4 |

### EXAMPLE 2

Manufacture of the pellet core by means of an extrusion method. Intermediate layer and retarding film are placed on the fluidised bed. The pellets used have a diameter of 710-1200 μm.

Composition [mg/DF]

| | | | | Film | |
|---|---|---|---|---|---|
| | | [mg/DF] | [mg/DF] | thickness | |
| | Ingredients | A | B | mg/cm² | Function |
| Pellet core | Hydromorphone-HCl | 16.0 | 16.0 | | Active substance |
| | Avicel PH 101 | 163.0 | 163.0 | | Extrusion adjuvant |
| | Methocel E5 | 1.0 | B | | Binding agent |
| Intermediate layer | Pharmacoat 606 | 3.6 | — | 0.5 | Film-forming agent |
| Retarding film | Ethyl cellulose | 4.5 | 4.5 | | Film-forming agent |
| | Dibutyl sebacate | 0.96 | 0.96 | 1.0 | Plasticiser |
| | Oleic acid | 0.54 | 0.54 | | Surfactant |
| | Silicon dioxide | 0.49 | 0.49 | | Release agent |

In-vitro Release Profile

Paddle apparatus according to the European Pharmacopoeia 2.9.3-1, 100 rpm, phosphate buffer pH 6.4 according to the USP, 900 ml, n=6

| Time (h) | Average value [%] A | Average value [%] B |
|---|---|---|
| 1 | 19.0 | 11.6 |
| 3 | 67.2 | 29.2 |
| 8 | 100.3 | 65.7 |

### EXAMPLE 3

Manufacture of the pellet core by means of an extrusion method. Intermediate layer and retarding film are placed on the fluidised bed. The pellets used have a diameter of 710-1200 μm.

Composition [mg/DF]

| | | | | Film | |
|---|---|---|---|---|---|
| | | [mg/DF] | [mg/DF] | thickness | |
| | Ingredients | A | B | mg/cm² | Function |
| Pellet core | Hydromorphone-HCl | 16.0 | 16.0 | | Active substance |
| | Avicel PH 101 | 163.0 | 163.0 | | Extrusion adjuvant |
| | Methocel E5 | 1.0 | 1.0 | | Binding agent |
| Intermediate layer | Pharmacoat 606 | 1.8 | — | 0.3 | Film-forming agent |
| Retarding film | Ethyl cellulose | 5.4 | 5.4 | | Film-forming agent |
| | Dibutyl sebacate | 1.15 | 1.15 | 1.0 | Plasticiser |
| | Oleic acid | 0.65 | 0.65 | | Surfactant |
| | Silicon dioxide | 0.59 | 0.59 | | Release agent |

In-vitro Release Profile

Paddle apparatus according to the European Pharmacopoeia 2.9.3-1, 100 rpm, phosphate buffer pH 6.4 according to the USP, 900 ml, n=6

| Time (h) | Average value [%] A | Average value [%] B |
|---|---|---|
| 1 | 9.6 | 4.3 |
| 3 | 49.7 | 9.2 |
| 8 | 88.6 | 31.1 |

### EXAMPLE 4

Manufacture of the pellet core by means of an extrusion method. Intermediate layer and retarding film are placed on the fluidised bed. The pellets used have a diameter of 710-1200 μm.

Composition [mg/DF]

| | | | | Film | |
|---|---|---|---|---|---|
| | | [mg/DF] | [mg/DF] | thickness | |
| | Ingredients | A | B | mg/cm² | Function |
| Pellet core | Amitriptyline HCl | 75.0 | 75.0 | | Active substance |
| | Ascorbic acid | 15.0 | 15.0 | | Stabiliser |
| | Avicel PH 101 | 138.8 | 138.8 | | Extrusion adjuvant |
| | Corn starch | 16.25 | 16.15 | | Filler |
| Intermediate layer | Povidon 25 | 2.45 | — | 1.7 | Binding agent |
| | Sodium carboxymethyl | 12.15 | — | | Swelling agent |
| | starch | | | | |
| Retarding film | Kollicoat SR (polymer) | 34.3 | 34.3 | 4.5 | Film-forming agent |
| | Triethyl citrate | 3.43 | 3.43 | | Plasticiser |
| | Povidon 30 | 5.48 | 5.48 | | Film-forming agent |
| | Syloid | 4.11 | 4.11 | | Release agent |

In-vitro Release Profile

Paddle apparatus according to the European Pharmacopoeia 2.9.3-1, 100 rpm, phosphate buffer pH 6.8 according to the USP, 900 ml, n=6

| Time (h) | Average value [%] A | Average value [%] B |
|---|---|---|
| 1 | 6.3 | 7.8 |
| 2 | 14.6 | 13.4 |
| 3 | 27.5 | 20.8 |
| 4 | 40.6 | 27.5 |
| 5 | 53.3 | 33.6 |
| 6 | 63.9 | 39.3 |
| 8 | 75.6 | 50.9 |
| 10 | 82.0 | 61.5 |
| 12 | 86.1 | 68.6 |

### EXAMPLE 5

Manufacture of the pellet core by means of an extrusion method. Intermediate layer and retarding film are placed on the fluidised bed. The pellets used have a diameter of 710-1200 μm.

Composition [mg/DF]

| | | | | Film | |
|---|---|---|---|---|---|
| | | [mg/DF] | [mg/DF] | thickness | |
| | Ingredients | A | B | mg/cm² | Function |
| Pellet core | Amitriptyline HCl | 75.0 | 75.0 | | Active substance |
| | Ascorbic acid | 15.0 | 15.0 | | Stabiliser |
| | Avicel PH 101 | 138.8 | 138.8 | | Extrusion adjuvant |
| | Corn starch | 16.25 | 16.15 | | Filler |
| Intermediate layer | Povidon 25 | 2.45 | — | 1.7 | Binding agent |
| | Sodium carboxymethyl | 12.15 | — | | Swelling agent |
| | starch | | | | |
| Retarding film | Kollicoat SR (polymer) | 39.2 | 39.2 | 4.5 | Film-forming agent |
| | Triethyl citrate | 3.92 | 3.92 | | Plasticiser |
| | Povidon 30 | 6.27 | 6.27 | | Film-forming agent |
| | Syloid | 4.70 | 4.70 | | Release agent |

In-vitro Release Profile Paddle apparatus according to the European Pharmacopoeia 2.9.3-1, 100 rpm, phosphate buffer pH 6.8 according to the USP, 900 ml, n=6

| Time (h) | Average value [%] A | Average value [%] B |
|---|---|---|
| 1 | 9.3 | 4.5 |
| 2 | 19.1 | 6.8 |
| 3 | 31.5 | 12.1 |
| 4 | 43.5 | 18.2 |
| 5 | 53.8 | 24.3 |
| 6 | 62.7 | 30.5 |
| 8 | 75.2 | 41.6 |
| 10 | 82.4 | 52.4 |
| 12 | 86.5 | 59.0 |

From the examples provided above, it is obvious that a clear increase in the release by the end of the release profile can be achieved through the use of a swellable intermediate layer according to the present invention. For this reason, the flattening of the release profile by the end can be overcome, and therefore an essentially complete release of the active substance can be achieved. The present examples are merely to illustrate the invention, but not to limit it.

1. A solid pharmaceutical formulation comprising a swellable intermediate layer that includes swellable material selected from the group consisting of starch, starch derivatives such as pregelatinised starch, sodium carboxyethyl starch, sodium starch gylcolate, cellulose, cellulose ethers such as HPMC, HPC, HEC, MC, sodium carboxymethyl cellulose, carrageenans, alginates, pectins, xanthanes and their derivatives, guar gum, tragacanth, polyvinyl pyrrolidones and mixtures therof; a substrate containing an active substance disposed within said intermediate layer; and a retarding coating layer on a side of said intermediate layer opposite said active substance, for the adjustment of a release profile for the active substance which essentially permits complete release of said active substance near the end of said release profile.

2. The pharmaceutical formulation according to claim 1, wherein the release profile is also initially retarded compared to that of the corresponding formulation without the intermediate layer.

3. The pharmaceutical formulation according to claim 1, wherein the release of said active substance after 8 hours, measured based on the rotating basket method according to European Pharmacopoeia 2.9.3-1, amounts to at least 75% of the total amount of the active substance contained in the formulation.

4. The pharmaceutical formulation according to claim 1, wherein the release amount of active substance after 8 hours, measured based on the rotating basket method according to the European Pharmacopoeia 2.9.3-1, amounts to at least 130% of that of the corresponding formulation without the intermediate layer.

5. The pharmaceutical formulation according to claim 1, wherein the release after 1 hour, measured based on the rotating basket method according to the European Pharmacopoeia 2.9.3-1 amounts between 5% and 35% of the total amount of the active substance contained in the formulation.

6. The pharmaceutical formulation according to claim 1, wherein the released amount of active substance after 1 hour, measured based on the rotating basket method according to the European Pharmacopoeia 2.9.3-1,amounts to between 70% and 120% of that which is released by the corresponding formulation without the intermediate layer.

7. The pharmaceutical formulation of claim 1, wherein the pharmaceutical formulation has the following release profile, measured based on the rotating basket method according the European Pharmacopoeia 2.9.3-1, as a percentage of the total amount of the active substance contained in the formulation:

| Time (h) | Percent |
|---|---|
| 1 | 5-35 |
| 3 | 45-75 |
| 8 | 85-100 |

8. The pharmaceutical formulation of claim 1, wherein the pharmaceutical formulation has the following release profile, measured based on the rotating basket method according to the European Pharmacopoeia 2.9.3-1, as a percentage of the total amount of the active substance contained in the formulation:

| Time (h) | Percent |
|---|---|
| 1 | 5-15 |
| 3 | 25-35 |
| 8 | 70-80 |
| 12 | 85-95 |

9. The pharmaceutical formulation of claim 1, wherein the peroral pharmaceutical formulation involves a formulation selected from the group that comprises tablets, capsules, pellets, microtablets, nanopellets, nanotablets, granulate, crystals as well as in capsules or sachets with pellets, microtablets, nanopellets, nanotablets, granulate and crystal.

10. The pharmaceutical formulation of claim 1, wherein the pharmaceutical formulation involves tablets, pellets, microtablets or capsules, preferably pellets or microtablets with a diameter (without coating) of 0.2 to 2.0 mm.

11. The pharmaceutical formulation of claim 1, wherein the swellable intermediate layer is applied in an amount of at least 0.01 mg/cm² to the substrate of the pharmaceutical formulation.

12. The pharmaceutical formulation of claim 1, wherein the intermediate layer is present in an amount of 0.01 to 10 mg/cm².

13. The pharmaceutical formulation of claim 1, wherein the swellable material is selected from sodium carboxyethyl starch, sodium starch glycolate, cellulose ethers, sodium carboxymethyl cellulose, polyvinyl pyrrolidones and mixtures thereof.

14. The pharmaceutical formulation of claim 1, wherein the cellulose ethers are selected from hydroxpropyl cellulose (HPMC), hydroxypropyl methyl cellulose (HPC), hydroxyethyl cellulose (HEC) and methyl cellulose (MC).

15. The pharmaceutical formulation of claim 1, wherein the intermediate layer also contains one or more adjuvants selected from binding agents, solvents, pH regulators, buffer substances, plasticizers, release agents and fillers.

16. The pharmaceutical formulation of claim 1, wherein the retarding coating layer includes a retarding polymer.

17. The pharmaceutical formulation of claim 16, wherein the retarding polymer is selected from the group that comprises cellulose ethers such as ethyl cellulose, insoluble polyacrylates such as Eudragit®NE, Eudragit®RL and polyvinyl acetates such as kollicoat SR30D.

18. The pharmaceutical formulation of claim 16, wherein the retarding coating layer also includes one or more adjuvants selected from the group that comprises binding agents, solvents, pH regulators, buffer substances, plasticizers, pore-forming agents, film-forming agents, breakdown agents, retarding agents; flavouring agents, dyestuffs, pigments, lubricants, release modifiers, release agents and fillers.

19. The pharmaceutical formulation of claim 1, wherein the active substance is selected from the group that comprises antihistamines, analgesics, non-steriodal anti-inflammatories, anti-emetics, antiepileptics, vasodilators, antitussive agents and expectorants, antiasthmatics, spasmolytics, agents for the treatment of diabetes, diuretics. Agents for the treatment of low blood pressure, agents for the treatment of high blood pressure, bronchodialators, steroids, antibiotics, anti-hemorrhoidal agents, hypnotics, psychotropic agents, antidepressants, agents for the treatment of diarrhea, mucolytics, sedatives, decongestants, laxatives, vitamins and stimulants.

20. The pharmaceutical formulation of claim 1, wherein the active substance is hydromorphone or amitriptyline or a pharmaceutically acceptable salt thereof.

21. A delayed-release, solid pharmaceutical formulation for peroral use, comprising a substrate that contains an active substance, a swellable intermediate layer and a retarding coating layer, wherein the swellable intermediate layer includes a swellable material that is selected from the group consisting of starch, starch derivatives such as pregelatinised starch, sodium carboxyethyl starch, sodium starch gylcolate, cellulose, HPC, HEC, MC sodium carboxymethyl cellulose, carrageenans, alginates, pectins xanthanes and their derivatives, guar gum, tragacanth, polyvinyl pyrrolidones and mixtures thereof, and which is available in an effective amount for the adjustment of a release profile for the active substance which profile is initially retarded compared to the release profile of the corresponding formulation without the intermediate layer and which essentially permits complete release of said active near the end of said release profile.

22. (canceled)
